(19) United States
(12) Patent Application Publication
(10) Pub. No.: US 2023/0293354 A1
(43) Pub. Date: Sep. 21, 2023

(54) Head Protection Device and Method of Manufacturing the Same

(71) Applicants: TAN TOCK SENG HOSPITAL PTE. LTD., Singapore (SG); SINGAPORE HEALTH SERVICES PTE LTD, Singapore (SG)

(72) Inventors: Jai Prashanth Rao, Singapore (SG); Sui Geok Karen Chua, Singapore (SG); Jun Cong Looi, Singapore (SG); Catherine Chia, Singapore (SG); Tegan K Plunkett, Singapore (SG)

(21) Appl. No.: 17/914,213

(22) PCT Filed: Mar. 23, 2021
(86) PCT No.: PCT/SG2021/050161

§ 371 Date: Sep. 23, 2022

(30) Foreign Application Priority Data
Mar. 25, 2020 (SG) 10202002777Q

(51) Int. Cl. A61F 13/12; B33Y 80/00; B33Y 50/00

(52) U.S. Cl. CPC A61F 13/12; B33Y 80/00; B33Y 50/00

(57) ABSTRACT

A customized head protection device comprising: a base plate shaped and dimensioned to shield a cranial defect area of a subject, the base plate comprising an cranial defect outline defining a first parameter around the cranial defect area, and a margin outline defining a second parameter around the first parameter, wherein the area between the first parameter and the second parameter defines an extension region adapted to fit onto a skin portion of the subject when worn. Also provided is a method of manufacturing the head protection device comprising identifying the cranial defect area of the subject, determining the aforementioned cranial defect and margin outlines, generating a 3D model object and 3D printing the device based on the 3D model object.

### TECHNICAL FIELD

The present disclosure relates to a head protection device and a method of manufacturing the device.

### BACKGROUND

The following discussion of the background is intended to facilitate understanding of the present disclosure. However, it should be appreciated that the discussion is not an acknowledgment or admission that any of the material referred to was published, known or a part of the common general knowledge in any jurisdiction as at the priority date of the application.

Neurosurgery typically involves the performance of craniotomies for exposure of the brain and intracranial contents. In post-operative intracranial hemorrhage or other complications, decompressive craniectomy may be used to alleviate elevated intracranial pressure.

Rigid ventilated helmets or rigid off the shelf protective helmets are typically used as temporary covers for post-decompression craniectomy (“DC”) subjects who have survived severe head injury, stroke, brain hemorrhages or brain tumours. These helmets provide temporary protection to the subjects until receipt of definitive cranioplasty procedures and customized skull implants, which can be several months later.

Use of such non-customized covers has been found to be non-ideal. This is in-part due to the discomfort caused by the weight of the helmets, heat and sweat retention when in use, and poor social cosmesis. In many cases, patient compliance is poor for the above reasons.

There is therefore a need for a head protection device that provides adequate customized rigid coverage of the skull defect that is feasible, safe to the skin, comfortable, acceptable and addresses one or more of the above problems.

### SUMMARY

A technical solution of the disclosure is the use of additive manufacturing, such as three-dimensional (3D) printing, to customize a head protection device. The head protection device may be regarded as prosthesis for external use.

The head protection device is envisaged to offer enhanced protection over the existing solutions. The head protection device is customizable, and envisaged to provide an acceptable level of comfort to the wearer.

It is envisaged that the head protection device can provide a temporary protection to a subject's skull bone defect prior to definitive surgery such as cranioplasty to replace the skull defect with an implant.

According with an aspect of the disclosure there is a customized head protection device comprising: a base plate shaped and dimensioned to shield a cranial defect area of a subject, the base plate comprising an cranial defect outline defining a first parameter around the cranial defect area, and a margin outline defining a second parameter around the first parameter, wherein the area between the first parameter and the second parameter defines an extension region adapted to fit onto a skin portion of the subject when worn.

In some embodiments, the outline offset may be approximately between 0.5 cm and 1.0 cm from and beyond the cranial defect outline. The outline offset may be set to about 1.5 cm±0.5 cm across known cases. Typically, the outline offset is determined by whether there is a presence of any bony anatomical features around the defect. It is envisaged that outline offset at different part of the head protection device may be varied to avoid the bony anatomical features.

In some embodiments, the plate has an interior surface, the interior surface comprises a contour generated towards a skin surface proximate the cranial defect area.

In some embodiments, the customized head protection device interior surface of the plate is at least 1 cm from the skin surface.

In some embodiments, the extension region comprises a plurality of attachment points shaped and dimensioned to receive corresponding attachment devices.

In some embodiments, the base plate comprises one or more ventilation holes. In some embodiments, the base plate may come in different shapes and sizes to accommodate different shapes and sizes of heads.

In some embodiments, the head protection device is made of a biocompatible material. The biocompatible material may be a lightweight biocompatible material. The biocompatible material may be at least one of Nylon, polyvinylchloride (PVC), polyethelyne, medical grade plastics, bone-like polyethelyne

According with another aspect there is a method of manufacturing a customized head protection device, the method comprising: (a.) identifying a cranial defect area of a subject; (b.) determining a cranial defect outline, the cranial defect outline forming a first parameter around the cranial defect area; (c.) determining a margin outline, the margin outline forming a second parameter around the cranial defect outline; wherein the area between the first parameter and the second parameter defines an extension region adapted to fit onto a skin portion of the subject when the head protection device is worn.

In some embodiments, the method further comprises the step of generating a 3D model object having a file format of at least one of a stereolithography (STL) and a virtual reality modelling language (VRML) file format.

Other aspects of the disclosure will be apparent to those of ordinary skill in the art upon review of the following description of specific embodiments of the disclosure in conjunction with the accompanying drawings.

### BRIEF DESCRIPTION OF THE DRAWINGS

Various embodiments are described, by way of example only, with reference to the accompanying drawings, in which:

FIG. 1 is a top view of an embodiment of a head protection device;

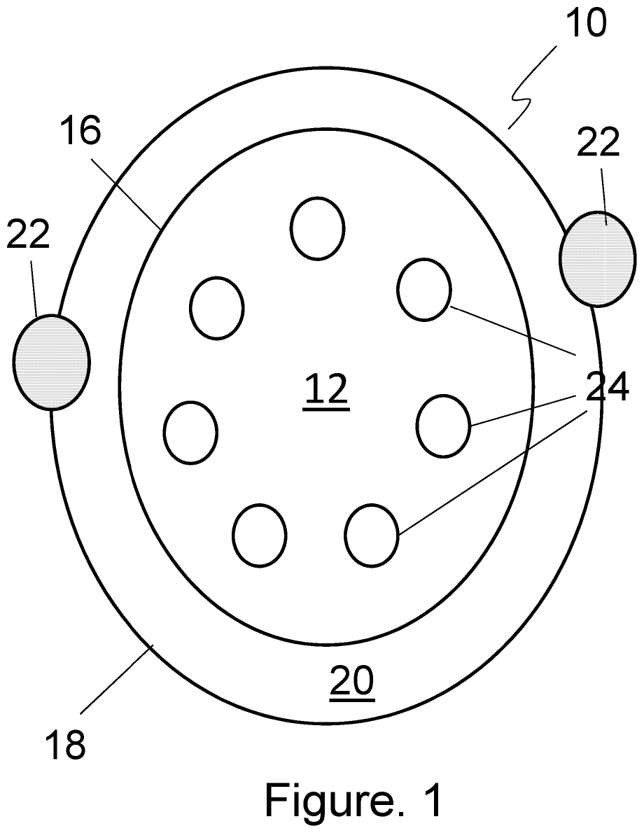

FIG. 2 is a perspective view of an embodiment of a head protection device;

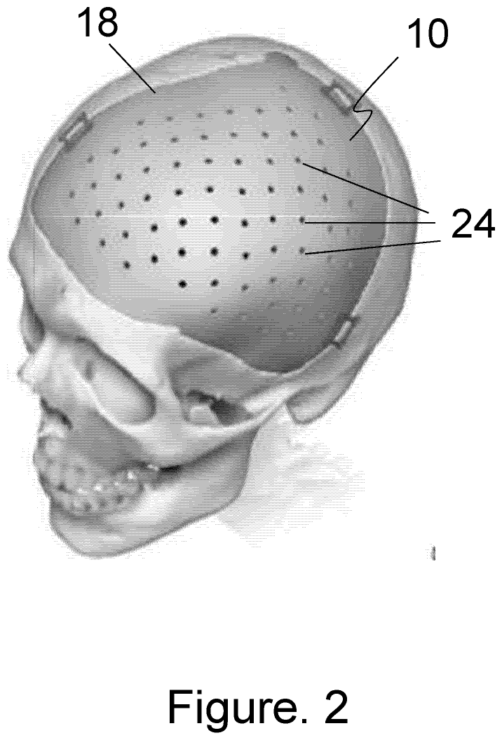

FIG. 3 shows a cranial defect outline of a subject (dark arrow, “Base outline”), an outline offset from and beyond the cranial defect outline (light arrow, “Margin”);

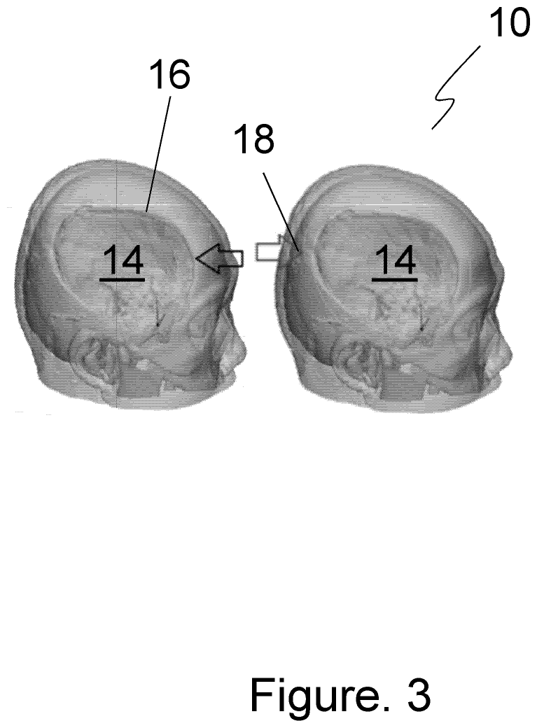

FIG. 4 shows an embodiment of the head protection device with through holes (“loops”) for receiving an attachment to the subject's head loops and ventilation holes; and

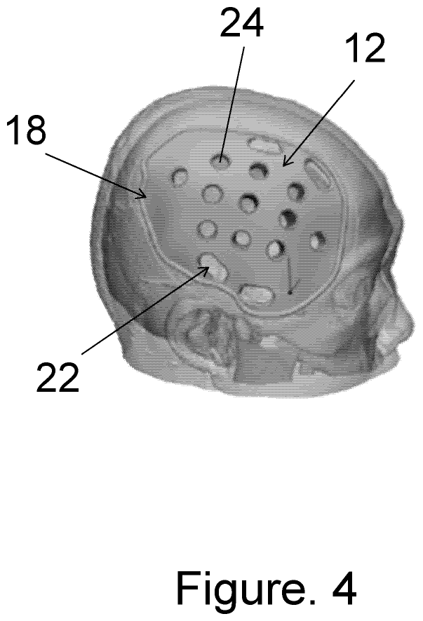

FIG. 5 is a flowchart depicting a method for manufacturing a customized head protection device according to some embodiments.

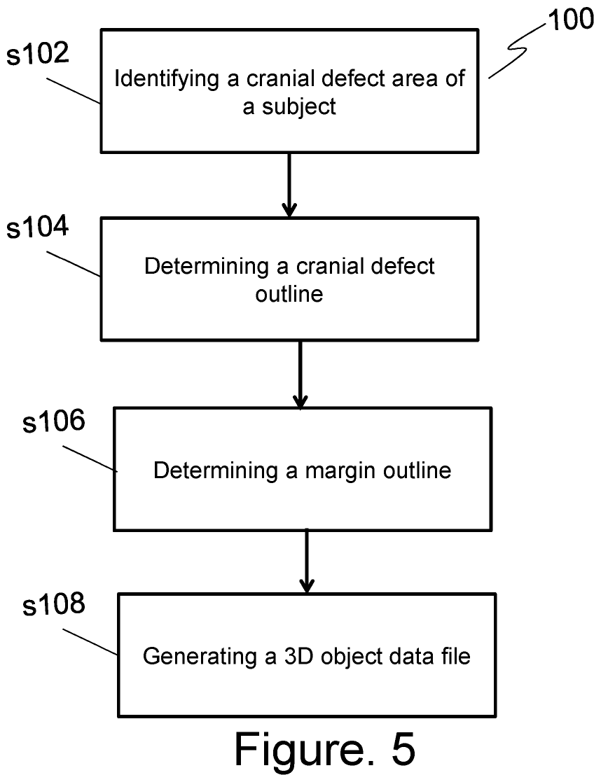

Other arrangements are possible and it is appreciable that the accompanying drawings are not to be understood as superseding the generality of the preceding description of the disclosure.

### DETAILED DESCRIPTION

Particular embodiments are described with reference to the accompanying drawings. The terminology used herein is for the purpose of describing particular embodiments only and is not intended to limit the scope of the present disclosure. Other definitions for selected terms used herein may be found within the detailed description of the disclosure and apply throughout the description. Additionally, unless defined otherwise, all technical and scientific terms used herein have the same meanings as commonly understood by one ordinary skilled in the art to which the present disclosure belongs. Where possible, the same reference numerals are used throughout the figures for clarity and consistency.

Throughout the specification, unless the context requires otherwise, the term “comprise” or variations such as “comprises” or “comprising”, will be understood to imply the inclusion of a stated integer or group of integers but not the exclusion of any other integer or group of integers.

Throughout the specification, unless the context requires otherwise, the term “include” or variations such as “includes” or “including”, will be understood to imply the inclusion of a stated integer or group of integers but not the exclusion of any other integer or group of integers.

Throughout the specification, unless the context requires otherwise, the term “have” or variations such as “has” or “having”, will be understood to imply the inclusion of a stated integer or group of integers but not the exclusion of any other integer or group of integers.

As used herein, the term ‘outline’ refers to a peripheral region or a parameter of an area.

As used herein, the term ‘cranial defect area’ refers to a deformity or defect of the skull of a mammalian subject. Such deformity or defect may be caused by various factors, such as, but not limited to, surgery.

As used herein, the term ‘additive manufacturing’ refers to an industry standard/process that comprises the step of joining/fusing/integrating materials to make objects. The term ‘3D printing’ refers to a specific example of additive manufacturing using 3D model data.

As used herein, certain parameters may be disclosed in a range format. It should be understood that the description in range format is merely for convenience and brevity and should not be construed as a limitation on the scope of the disclosed range. Accordingly, the description of a range should be considered to have specifically disclosed all the possible sub-ranges as well as individual numerical values within that range. In addition, ranges may include decimal places of up to three decimal places.

As used herein, the term ‘about’ may include +/−10% of the parameter or value.

According with an embodiment of the disclosure there is a head protection device 10 comprising a base plate 12 shaped and dimensioned to shield a cranial defect area 14 of a subject. The subject may be a mammalian subject, such as a human being.

The base plate 12 may have an outer surface shaped and dimensioned in the form of at least a portion of a healthy skull of a subject. In some embodiments, the outer surface mimics a healthy portion of the healthy skull of the subject. The healthy portion may correspond with a healthy side of the subject's skull. The healthy side may be at least one of a left side, a right side, a top side and a bottom side. The outer surface may be a mirror image of the healthy side.

The base plate 12 is typically a customizable base plate 12 that can vary according to different subject's head and/or cranial defect area 14. The customizable base plate 12 takes into account the following parameters for customization:
a. a cranial defect outline 16 surrounding the cranial defect area 14; and
b. a outline offset/parameter 18 beyond and larger than the cranial defect outline 16. The outline offset/parameter 18 may also be referred to as a margin outline.

The area between the cranial defect online 16 and the margin outline 18 may be regarded as an extension portion 20 of the base plate. The extension portion 20 may also be referred to as a margin area.

In consideration of the offset parameter 18, care is taken to provide sufficient extension portion 20 for the base plate 12 to rest on a skin portion proximate (above) a boney area surrounding the defect area. The extension portion 20 is shaped and dimensioned to minimize any pressure build up on the defect area that may be caused by wearing the device 10.

It is contemplated that the margin outline 18 of the device 10 is shaped to follow an outer contour of a healthy head, including the skull and skin of a subject.

In customizing the head protection device 10, the cranial defect outline 16 may be between about 0.5 centimeters (cm) to about 1 cm wider than the cranial defect area. The margin outline 18 may be between 1.5 cm±0.5 cm from the cranial defect outline 16. The margin outline may vary at different parts of the base plate 12 as it is constrained by the features around the defect which must be avoided. For example, at areas closer to the frontal portion of the subject (e.g. face), the margin outline may need to be set to 0.5 cm or less to avoid close contact with the eyebrow ridge (supraorbital ridge) region, so to avoid creating a pressure point on the skin when the head protection device 10 is worn.

In some embodiments, the interior surface of the base plate 12 comprises a secondary contour generated towards a skin surface of the subject head. Such an arrangement preferably maintains a gap between the head protection device 10 and skin within the cranial defect outline 16, further mitigating any pressure exerted on the cranial defect area by the head protection device 10.

When worn, a gap may be maintained between the secondary contour and the skin surface. In some embodiments, the gap may be between 0.5 cm to 1.5 cm. In some embodiments, an average of a 1 cm gap between the secondary contour and the skin surface may be maintained.

One or more attachment points 22 may be created within the margin area 20. The one or more attachment points may be in the form of through holes or loops for the looping of fasteners such as elastic bands for attachment to the subject's head.

It is contemplated that other fasteners or attachment mechanisms may be adopted.

A plurality of through holes 24 may be created on the base plate 12. Such plurality of through holes may be once used as dura suture holes for use in any cranial implant, and may serve an additional purpose as ventilation holes on the head protection device 10. Each of the plurality of through holes 24 may be suitably spaced apart from another through holes 24.

At least a portion of the base plate 12 may be formed from biocompatible material, preferably lightweight biocompatible material, such as, Nylon, Polyvinylchloride (PVC), Polyethelyne, medical grade plastics, bone-like polyethelyne, etc.

According to another aspect of the disclosure there is a method 100 of manufacturing a customized head protection device. The method of manufacturing may be suitably implemented using additive manufacturing technology such as 3D printing technology. A 3D model data may be suitably generated as input to a 3D printer for printing. The 3D model data may be in the form of at least one of a stereolithography (STL) file format and a virtual reality modeling language (VRML) file format.

The method 100 includes the step of identifying a cranial defect area of a subject (step s102); determining a cranial defect outline (step s104), the cranial defect outline forming a first parameter around the cranial defect area; determining a margin outline, the margin outline forming a second parameter around the cranial defect outline (step s106); and wherein the area between the first parameter and the second parameter defines an extension region adapted to fit onto a skin portion of the subject when the head protection device 10 is worn.

It is contemplated that the distance between the first parameter and the second parameter defining the extension region may not be equally spaced throughout.

The method 100 may be implemented as software code for generating a 3D model data. The software code may be suitably executed by a computer processor (examples include distributed or local computer, servers) configured or arrange in data communication with a 3D printer. After step s106, the method 100 suitably generates a 3D object data file (step s108) for 3D printing. The method 100 may include the loading of suitable biocompatible materials for the purpose of 3D printing (step s110). Optionally, pre-processing or post-processing steps (not shown) may be performed.

The method 100 may further comprise a step of acquiring a medical image of the cranial defect area of the subject. The medical image may be used for the determination of the cranial defect outline, the margin outline, and the extension region, before a step of 3D printing the customized head protection device via a 3D printer.

The medical image can be acquired via various medical imaging techniques. Such medical imaging techniques may include X-rays, Computed

Tomography (CT) scan, Magnetic Resonance Imaging (MRI), positron-emission tomography (PET).

It is to be appreciated by the person skilled in the art that variations and combinations of features described above, not being alternatives or substitutes, may be combined to form yet further embodiments falling within the intended scope of the invention.

As would be understood by a person skilled in the art, each embodiment may be used in combination with other embodiment or several embodiments.

1. A customizable head protection device comprising:
a base plate shaped and dimensioned to shield a cranial defect area of a subject, the base plate comprising an cranial defect outline defining a first parameter around the cranial defect area, and a margin outline defining a second parameter around the first parameter, wherein the area between the first parameter and the second parameter defines an extension region adapted to fit onto a skin portion of the subject when worn.

2. The customizable head protection device of claim 1, wherein the outline offset may be approximately between 0.5 cm and 1.0 cm from and beyond the cranial defect outline.

3. The customizable head protection device of claim 1, wherein the plate has an interior surface, the interior surface comprises a contour generated towards a skin surface proximate the cranial defect area.

4. The customizable head protection device of claim 3, wherein when positioned, the interior surface of the plate is at least 1.0 cm from the skin surface.

5. The customizable head protection device of claim 1, wherein the extension region comprises a plurality of attachment points shaped and dimensioned to receive corresponding attachment devices.

6. The customizable head protection device of claim 1, wherein the plate comprises one or more ventilation holes.

7. The customizable head protection device of claim 1, wherein the head protection device is made of a biocompatible material.

8. The customizable head protection device of claim 7, wherein the biocompatible material is a lightweight biocompatible material.

9. The customizable head protection device of claim 7, wherein the biocompatible material is at least one of Nylon, polyvinylchloride (PVC), polyethelyne, medical grade plastics, bone-like polyethelyne.

10. A method of manufacturing a head protection device, the method comprising:
(a.) identifying a cranial defect area of a subject;
(b.) determining a cranial defect outline, the cranial defect outline forming a first parameter around the cranial defect area;
(c.) determining a margin outline, the margin outline forming a second parameter around the cranial defect outline;
wherein the area between the first parameter and the second parameter defines an extension region adapted to custom-fit onto a skin portion of the subject when the head protection device is worn.

11. The method of claim 10, further comprises the step of generating a 3D model object having a file format of at least one of a stereolithography (STL) and a virtual reality modeling language (VRML) file format.

12. The method of claim 11, further comprising a step of acquiring a medical image of the cranial defect area of the subject before the step of determining the cranial defect outline.

13. The method of claim 12, wherein the medical image is acquired via a CT scan.

14. The method of claim 11, further comprises the step of 3D printing the customized head protection device based on the 3D model object.

15. The customizable head protection device of claim 3, wherein when positioned, an edge of the plate is in apposition with the skin surface.
